SEQUENCE LISTING (1 sequence)

SEQ ID NO: 1 - DNA, 20 bp, Homo sapiens
  source 1..20: mol_type = genomic DNA
cagtatcaggatccaacaag

(19) United States
(12) Patent Application Publication
(10) Pub. No.: US 2026/0226413 A1
(43) Pub. Date: Aug. 6, 2026

(54) METHODS OF TARGETING GLYCOGEN SYNTHASE KINASE 3 BETA IN NK CELLS

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Meisam NAEIMI KARAROUDI, Columbus, OH (US); Marcelo DE SOUZA FERNANDES PEREIRA, Columbus, OH (US)

(21) Appl. No.: 19/148,987

(22) PCT Filed: Jan. 17, 2024
(86) PCT No.: PCT/US2024/011831

§ 371 Date: Jul. 17, 2025

Related U.S. Application Data
(60) Provisional application No. 63/439,426, filed on Jan. 17, 2023.

(51) Int. Cl. C12N 5/0783; A61K 35/17; A61P 35/00; C12N 9/12; C12N 9/22; C12N 15/11

(52) U.S. Cl. CPC C12N 5/0646; A61K 35/17; A61P 35/00; C12N 9/12; C12N 9/226; C12N 15/111; C12N 2310/20; C12N 2501/2302; C12N 2502/99; C12N 2510/00; C12Y 207/11026

(57) ABSTRACT

The present disclosure relates methods of engineering natural killer cells for treating, preventing, inhibiting, decreasing, and/or ameliorating diseases, including but not limited to cancer and other proliferative diseases.

### RELATED APPLICATION

This PCT application claims priority to, and the benefit of, U.S. Provisional Patent Application No. 63/439,426, filed Jan. 17, 2023, entitled “TARGETING GLYCOGEN SYNTHASE KINASE 3 BETA IN NK CELLS FOR ENHANCED ANTITUMOR ACTIVITY,” which is incorporated by reference herein in its entirety.

### REFERENCE TO SEQUENCE LISTING

The sequence listing submitted on Jan. 17, 2024, as an .XML file entitled “10935-026WO1.XML” created on Jan. 16, 2024, and having a file size of 2,010 bytes is hereby incorporated by reference pursuant to 37 C.F.R. § 1.52(e)(5).

### FIELD

The present disclosure relates methods of genetically modifying natural killer cells for treating, preventing, inhibiting, decreasing, reducing and/or ameliorating diseases, including but not limited to cancer and other proliferative diseases.

### BACKGROUND

The antitumor activity of NK cells has been widely demonstrated for multiple cancer types. This antitumor role can be limited by several factors in the tumor microenvironment that mediate metabolic suppression. As a result, chemical inhibitors, genetic, and epigenetic alterations are now being tested to overcome these suppressive mechanisms in NK cells. One promising target to enhance NK cell cytotoxic activity was GSK3β, a serine threonine kinase. GSK3β has been shown to regulates multiple functions in other cell types, but its role in lymphocytes including NK cells has not been well studied. In a few reports, drug inhibition of GSK3β has been shown to improve maturation and antitumor activity of NK cells with elevated GSK3β such as IL-15 expanded NK cells or NK cells isolated from AML patients. However, a specific anticancer GSK3β inhibitor has yet been developed. Given this limitation, there remains a need to develop a cancer therapeutic or methods thereof for inhibiting GSK3β activity to promote antitumor activity.

### SUMMARY

The present disclosure provides methods of generating glycogen synthase kinase 3 beta (GSK3β) deficient natural killer (NK) cells comprising enhanced cellular metabolism and enhanced antitumor activity. The present disclosure also provides methods of treating, preventing, inhibiting, decreasing, reducing, and/or ameliorating a cancer in a subject by administering a composition comprising a GSK3β deficient NK cell, wherein the GSK3β deficient NK cell comprises enhanced cellular metabolism and enhanced antitumor activity.

In one aspect, disclosed herein are methods of generating a glycogen synthase kinase 3 beta (GSK3β) deficient natural killer (NK) cell, the method comprising isolating the NK cell, deleting GSK3β in the NK cell using a gene editing system, and expanding and stimulating the NK cell with feeder cells comprising membrane bound (mb) IL21 on the surface of the feeder cells, wherein the GSK3β deficient NK cell comprises decreased GSK3β expression and increased cellular metabolism relative to a control.

In one aspect, disclosed herein are methods of treating, preventing, inhibiting, decreasing, reducing and/or ameliorating a cancer and/or metastasis (such as, for example acute myeloid leukemia, chronic myelogenous leukemia, medulloblastoma, osteosarcoma, or glioblastoma), the method comprising administering to the subject a composition comprising a glycogen synthase kinase 3 beta (GSK3β) deficient natural killer (NK) cell and a pharmaceutically acceptable carrier, wherein the GSK3β deficient NK cell comprises increased cellular metabolism and increased antitumor activity.

Also disclosed herein are methods of generating a GSK3β deficient NK cell any preceding aspect or methods of treating, preventing, inhibiting, decreasing, reducing and/or ameliorating a cancer and/or metastasis of any preceding aspect, wherein the methods uses a gene editing system, including but not limited to CRISPR/Cas9 gene editing systems to decrease and/or delete GSK3β in the NK cell. In some embodiments, the method of any preceding aspect comprises introducing a Cas9/ribonucleoprotein (RNP) complex into the NK cell.

In one aspect, disclosed herein are methods of generating a GSK3β deficient NK cell any preceding aspect or methods of treating, preventing, inhibiting, decreasing, reducing and/or ameliorating a cancer and/or metastasis of any preceding aspect, wherein the feeder cells further comprise membrane bound 4-1BBL. In some embodiments, the GSK3β deficient NK cell is stimulated with feeder cells at least once per week. In some embodiments, the GSK3β deficient NK cell is stimulated with feeder cells for at least two weeks. In some embodiments, the feeder cells are K562 feeder cells or the feeder line comprises CTSX-002. In some embodiments, the GSK3β deficient NK cells are supplemented with at least 50IU of an IL-2. In some embodiments, the IL-2 is a human recombinant IL-2 (rIL-2).

Also disclosed are methods of generating a GSK3β deficient NK cell any preceding aspect or methods of treating, preventing, inhibiting, decreasing, reducing and/or ameliorating a cancer and/or metastasis of any preceding aspect, whereinGSK3β expression or activity is inhibited in the NK cell. In some embodiments, the GSK3β deficient NK cell comprises increased mitochondrial metabolism and glycolytic metabolism. In some embodiments, the GSK3β deficient NK cell comprises increased cytotoxicity of the GSK3β deficient NK cell.

In one aspect, disclosed herein is a natural killer (NK) cell modified by any one of methods of any preceding aspect.

In one aspect, disclosed herein is a GSK3β deficient NK cell comprising decreased expression of GSK3β and increased mitochondrial metabolism and increased glycolytic metabolism.

### BRIEF DESCRIPTION OF FIGURES

The accompanying figures, which are incorporated in and constitute a part of this specification, illustrate several aspects described below.

FIGS. 1A, 1B, and 1C show the generation of GSK3β-KO FC-21 expanded NK cells. FIG. 1A shows the GSK3β expression after expansion on FC-21 was evaluated by RNA-seq analysis. FIGS. 1B and 1C show the efficiency of Cas9/RNP mediated deletion of GSK3 was evaluated by western blot and RNA-seq. Also see FIG. 6.

FIGS. 2A, 2B, 2C, 2D, and 2E show the deletion of GSK3β did not alter antitumor activity or maturation of FC-21 expanded NK cells. FIGS. 2A and 2B show both WT and GSK3β-KO NK cells were cocultured with AML tumor cell lines for 4 hours (FIG. 2A shows Kasumi1 tumor cell line and FIG. 2B shows HL60 tumor cell line). NK cell killing is shown as percentage of cell lysis (n=3). FIG. 2C shows IL2, IFNγ, and TNFα release was assessed after 4 hours of PHA stimulation of WT and GSK3β-KO NK cells (n=3). FIG. 2D shows CD57 expression was evaluated by flow cytometry on FC-21 expanded WT and GSK3β-KO NK cells (n=6). FIG. 2E shows B3GAT1 expression in WT, FC-21 expanded WT, and FC-21 expanded GSK3β-KO NK cells as measured by RNA-seq. ***p≤0.001. Also see FIG. 7.

FIG. 3 shows the GSK3β-KO in NK resulted in transcriptomic change. RNA-seq analysis was performed on NK cells from FC-21 expanded WT and GSK3β-KO. FIG. 3 also shows a volcano plat representing DEG (WT/KO), GO enrichment (WT), and RNA-seq data showing the top downregulated and top upregulated genes affected by GSK3β deletion. *p≤0.05, **p≤0.01, ***p≤0.001, ****p≤0.0001.

FIGS. 4A, 4B, 4C, 4D, 4E, 4F, 4G, and 4H show that deletion of GSK3β enhances metabolic capacity of NK cells. FIGS. 4A and 4B shows mitochondrial and glycolytic stress, respectively, which is summarized data of metabolic analysis of paired WT and GSK3β-KO NK cells (n=3; mean±SD). FIG. 4C shows OCR/ECAR ratio. FIGS. 4D and 4E shows the maximum respiratory capacity and reserve capacity derived from FIG. 4A. FIGS. 4F, 4G, and 4H shows the glycolysis, glycolytic capacity, and glycolytic reserve derived from FIG. 4B. N=4, *p≤0.05, **p≤0.01, *** p≤0.001, ****p≤0.0001.

FIG. 5 shows the downregulated genes identified in GSK3β-KO NK cells were mostly upregulated in GSK3β-overexpressing NK cells from patients with AML. RNA-seq analysis was performed on NK cells at different stages of maturation isolated by sorting from healthy donors and patients with AML. *p≤0.05.

FIG. 6 shows the efficiency of Cas9/RNP mediated deletion of GSK3β was evaluated by western blot.

FIGS. 7A, 7B, 7C, and 7D show the NK cell killing of (A) K452-3 donors, (B) U373 (glioblastoma)-3 donors, (C) DAOY (medulloblastoma), and (D) MG63 (osteosarcoma)-3 donors.

FIG. 8 shows the effect of GSK3β-KO on expression of RelA, RelB, Rel, NFKB1, NFKB2, and ITGAL.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, palliatively, or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

As used herein, the term “genetically modified” refers to a living cell, tissue, or organism whose genetic material has been altered using genetic engineering techniques. The genetic modification results in an alteration that does not occur naturally by mating and/or natural recombination. Modified genes can be transferred within the same species, across species (creating transgenic organisms), and across kingdoms. New, exogenous genes can be introduced, or endogenous genes can be enhanced, altered, or knocked out.

As used herein, a “mutation” refers to changing the structure of a gene, resulting in a variant form that may be transmitted to later generations. A mutation is caused by the alteration of single nucleotides in DNA, or the deletion, insertion, or rearrangement of larger sections of genes. A mutation can lead to the expression of a protein that has been changed physically or functionally leading to lethality, non-lethal dysfunction effects, or no effects.

As used herein, the term, “deletion,” also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome.

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Methods

The present disclosure provides methods of generating glycogen synthase kinase 3 beta (GSK3β) deficient natural killer (NK) cells comprising enhanced cellular metabolism and enhanced antitumor activity. The present disclosure also provides methods of treating, preventing, inhibiting, decreasing, reducing, and/or ameliorating a cancer in a subject by administering a composition comprising a GSK3β deficient NK cell, wherein the GSK3β deficient NK cell comprises enhanced cellular metabolism and enhanced antitumor activity.

In one aspect, disclosed herein is a method of generating a glycogen synthase kinase 3 beta (GSK3β) deficient natural killer (NK) cell, the method comprising isolating the NK cell, deleting GSK3β in the NK cell using a gene editing system, and expanding and stimulating the NK cell with feeder cells comprising membrane bound (mb) IL21 on the surface of the feeder cells, wherein the GSK3β deficient NK cell comprises decreased GSK3β expression and increased cellular metabolism relative to a control.

In one aspect, disclosed herein is a method of treating, preventing, inhibiting, decreasing, reducing and/or ameliorating a cancer and/or metastasis (such as, for example acute myeloid leukemia, chronic myelogenous leukemia, medulloblastoma, osteosarcoma, or gliobastoma), the method comprising administering to the subject a composition comprising a glycogen synthase kinase 3 beta (GSK3β) deficient natural killer (NK) cell and a pharmaceutically acceptable carrier, wherein the GSK3β deficient NK cell comprises increased cellular metabolism and increased antitumor activity.

It is understood and herein contemplated that the disclosed methods can be utilized with any cell type including natural killer cells (NK cells), T cells, B cells, macrophages, fibroblasts, osteoblasts, hepatocytes, neuronal cells, epithelial cells, and/or muscle cells. Human NK cells are a subset of peripheral blood lymphocytes defined by the expression of CD56 or CD16 and the absence of T cell receptor (CD3). NK cells sense and kill target cells that lack major histocompatibility complex (MHC)-class I molecules. NK cell activating receptors include, among others, the natural cytotoxicity receptors (NKp30, NKp44 and NKp46), and lectin-like receptors NKG2D and DNAM-1. Their ligands are expressed on stressed, transformed, or infected cells but not on normal cells, making normal cells resistant to NK cell killing. NK cell activation is negatively regulated via inhibitory receptors, such as killer immunoglobin (Ig)-like receptors (KIRs), NKG2A/CD94, TGFα, and leukocyte Ig-like receptor-1 (LIR-1).

Also disclosed herein are methods of generating a GSK3β deficient NK cell any preceding aspect or methods of treating, preventing, inhibiting, decreasing, reducing and/or ameliorating a cancer and/or metastasis of any preceding aspect, wherein the methods uses a gene editing system, including but not limited to CRISPR/Cas9 gene editing systems to decrease and/or delete GSK3β in the NK cell. In some embodiments, the method of any preceding aspect comprises introducing a Cas9/ribonucleoprotein (RNP) complex into the NK cell. In general, “CRISPR system”, “CRISPR gene editing system”, or “CRISPR integration system” refers collectively to transcripts and other elements involved in the expression of or directing the activity of CRISPR-associated “Cas” genes. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly, the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis.

To make the RNP complex, crRNA and tracrRNA can be mixed at a 1:1, 2:1, or 1:2 ratio of concentrations between about 50 μM and about 500 μM (for example, 50 μM, 60 μM, 70 μM, 80 μM, 90 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, or 500 μM), preferably between 100 μM and about 300 μM, most preferably about 200 μM at 95° C. for about 5 min to form a crRNA: tracrRNA complex (i.e., the guide RNA). The crRNA: tracrRNA complex can then be mixed with between about 20 μM and about 50 μM (for example 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, or 50 μM) final dilution of a Cas endonuclease (such as, for example, Cas9).

Once bound to the target sequence in the target cell, the CRISPR loci can modify the genome by introducing into the target DNA, such as, for example GSK3β, insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof. Thus, the disclosed methods can be used to generate knock-outs, or knock-ins when combined with DNA for homologous recombination. It should be understood that transduction of CRISPR elements to the target DNA can be achieved using viral or non-viral approaches.

In non-viral approaches, one or more elements of the CRISPR system are electroporated into the NK cells. In some embodiments, the method of any preceding aspect comprises introducing a Cas9/ribonucleoprotein (RNP) complex into the NK cell. In some embodiments, the method of any preceding aspect comprises electroporated a Cas9/ribonucleoprotein (RNP) complex into the NK cell.

It is also noted that NK cell activation is negatively regulated via inhibitory receptors, such as killer immunoglobulin (Ig)-like receptors (KIRs), NKG2A/CD94, TGFβ, and leukocyte Ig-like receptor-1 (LIR-1). Engagement of one inhibitory receptor may be sufficient to prevent target lysis. Hence NK cells efficiently target cells that express many stress-induced ligands, and few MHC class I ligands. TGFβ is a major immunosuppressive cytokine which inhibits the activation and functions of NK cells. Thus, it is understood and herein contemplated that one modification of NK cells, such as for, example GSK3 deficient NK cells, would be advantageous is the suppression of inhibitory receptors, such as killer immunoglobin (Ig)-like receptors (KIRs), NKG2A/CD94, TGFβ, and leukocyte Ig-like receptor-1 (LIR-1) so the negative regulation of NK cells would be suppressed. Such modified cells would be very useful in immunotherapy of any disease or condition that could be treated with the addition of NK cells. Thus, in one aspect, disclosed herein are deficient NK cells comprising a knockout of the gene encoding the GSK3β.

The method disclosed herein also comprises incubating the NK cell in a media suitable for the propagation of NK cells. It is understood and herein contemplated that the culturing conditions can comprise the addition of cytokines, antibodies, and/or feeder cells. Thus, in one aspect, disclosed herein are methods of genetically modifying an NK cell, further comprising incubating the NK cells for 1, 2, 3, 4, 5, 6, 7, 8, 9, 10, 11, 12, 13, or 14 days prior to transducing the cells in media that supports the propagation of NK cells; wherein the media further comprises cytokines, antibodies, and/or feeder cells. For example, the media can comprise IL-2, IL-12, IL-15, IL-18, and/or IL-21.

In one aspect, the feeder cells can be purified from feeder cells that stimulate NK cells. NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. The It is understood and herein contemplated that the period of culturing can be between 1 and 14 days post electroporation (i.e, 1, 2, 3, 4, 5, 6, 7, 8, 9, 10, 11, 12, 13, or 14 days), preferably between 3 and 7 days, most preferably between 4 and 6 days.

In one aspect, disclosed herein are methods of generating a GSK3β deficient NK cell any preceding aspect or methods of treating, preventing, inhibiting, decreasing, reducing and/or ameliorating a cancer and/or metastasis of any preceding aspect, wherein the feeder cells further comprise membrane bound 4-1BBL. In some embodiments, the GSK3β deficient NK cell is stimulated with feeder cells at least once per week. In some embodiments, the GSK3β deficient NK cell is stimulated with feeder cells for at least two weeks. In some embodiments, the feeder cells are K562 feeder cells. In some embodiments, the feeder line comprises CTSX-002. In some embodiments, the GSK3β deficient NK cells are supplemented with at least 50IU of an IL-2. In some embodiments, the IL-2 is a human recombinant IL-2 (rIL-2).

As used herein, “glycogen synthase kinase 3B (GSK3β) activity” refers to the addition of a phosphoryl moiety to key enzymes of various cellular processes including, but not limited to glycogen/glucose metabolism, gene regulation, cellular proliferation, and cell cycle pathways. It should be noted that the addition/removal of phosphate groups (also termed phosphorylation/dephosphorylation) can drastically impact said cellular processes. A non-limiting example of GSK3β activity is the phosphorylation of glycogen synthase (GS), which inhibits GS from synthesizing glycogen polymers, and promotes release of glucose molecules leading to hyperglycemia. Because GSK3β is a central hub for cellular metabolism, it has been characterized as a contributor to numerous diseases, including but not limited to cancer, diabetes, obesity, and Alzheimer's disease. Also disclosed are methods of generating a GSK3β deficient NK cell any preceding aspect or methods of treating, preventing, inhibiting, decreasing, reducing and/or ameliorating a cancer and/or metastasis of any preceding aspect, whereinGSK3β expression or activity is inhibited in the NK cell. In some embodiments, the GSK3β deficient NK cell comprises increased mitochondrial metabolism and glycolytic metabolism.

As noted throughout the present disclosure, the disclosed modified NK cells are ideally suited for use in immunotherapy such as the adoptive transfer of modified (i.e, engineered NK cells) to a subject in need thereof. Thus, in one aspect, disclosed herein are methods of adoptively transferring an engineered NK cells to a subject in need thereof said method comprising a) obtaining a target NK cell to be modified; b) deleting, decreasing, and/or reducing GSK3β using a gene editing system of any preceding aspect; c) expanding and stimulating the NK cell with feeder cells comprising membrane bound (mb) IL21 on the surface of the feeder cells; and d) transferring the engineered NK cell into the subject, wherein the modified NK cell comprises decreased GSK3β expression and increased mitochondrial metabolism and increased glycolytic metabolism. In some embodiments, the GSK3β deficient NK cell comprises increased cytotoxicity of the GSK3β deficient NK cell.

The disclosed method comprises GSK3β deficient NK cells that can be expanded and stimulated prior to administration of the modified (i.e., engineered) NK cells to the subject. For example, disclosed herein are methods of adoptively transferring NK cells to a subject in need thereof wherein the NK cell is expanded with mbIL-21 expressing feeder cells prior to administration to the subject. In some aspects, it is understood and herein contemplated that the stimulation and expansion of the modified (i.e., engineered) NK cells can occur in vivo following or concurrent with the administration of the modified NK cells to the subject. Accordingly disclosed herein are immunotherapy methods wherein the NK cells are expanded in the subject following transfer of the NK cells to the subject via the administration of IL-21 or irradiated mbIL-21 expressing feeder cells.

It is understood and herein contemplated that the disclosed modified NK cell and adoptive transfer methods of the modified NK cells can be effective immunotherapy against a cancer. The disclosed methods and compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, disclosed herein is a natural killer (NK) cell modified by any one of methods of any preceding aspect.

In one aspect, the disclosed methods generate and/or use a GSK3β deficient NK cell to treat, prevent, inhibit, decrease, reduce and/or ameliorate a cancer and/or metastasis (such as, for example acute myeloid leukemia, chronic myelogenous leukemia, medulloblastoma, osteosarcoma, or glioblastoma), wherein the GSK3β deficient NK cell comprises decreased expression and/or activity of GSK3β, increased mitochondrial metabolism, and increased glycolytic metabolism.

In some embodiments, the method of any preceding aspect comprising administering a composition comprising the GSK deficient NK cell and a pharmaceutical acceptable carrier.

The compositions disclosed herein can also be administered in vivo in a pharmaceutically acceptable carrier. By “pharmaceutically acceptable” means a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be delivered directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Targeting GSK3β in NK Cells for Enhanced Antitumor Activity

Loss of cytotoxicity and defective metabolism are linked to increases in glycogen synthase kinase 3 beta (GSK3β) in natural killer (NK) cells from patients with acute myeloid leukemia or from healthy donors after expansion ex vivo with IL-15. Herein, expansion of NK cells with feeder cells expressing membrane-bound IL-21 did not alter GSK3β levels. GSK3β was deleted using Cas9/RNP approach and the paired-donor knockout and wild-type (WT) NK cells were expanded and assessed for transcriptional and functional alterations induced by loss of GSK3β. GSK3β-KO cells demonstrated significant changes in expression of genes related to rRNA processing, cell proliferation, and metabolic function, indicating metabolic reprogramming. Cellular energetics measured and GSK3β-KO NK cells exhibited 150% higher spare respiratory capacity, a marker of metabolic fitness. This is particularly important as pervious reports on NK cells expanded by IL 15 showed upregulation of GSK and a need for using drug inhibitors with lower on-target effects. The present disclosure teaches a novel benefit of using mbIL21 expansion of NK cells and gene GSK3β in these cells.

The disclosed technology can be used instead of non-specific GSK3β inhibitors. It demonstrated the superior benefit of using IL21 expressing feeder cells over IL 15 based expansions. The GSK3β-KO cells can be used to treat several cancers.

### Example 2: Deletion of Glycogen Synthase Kinase 3 Beta Reprograms NK Cell Metabolism

Drug inhibition of GSK3β in NK cells improves their maturation and cytotoxic activity, but the mechanisms of GSK3β-mediated dysfunction have not been well studied. Herein, expansion of NK cells with feeder cells expressing membrane-bound IL-21 did not alter GSK3β levels, allowing for studying of GSK3β function using CRISPR gene editing. GSK3β was deleted and paired-donor knockout and wild-type (WT) NK cells were expanded and assessed for transcriptional and functional alterations induced by loss of GSK3β. Surprisingly, the data show that deletion of GSK3β did not alter cytotoxicity, cytokine production, or maturation (as determined by CD57 expression). However, GSK3β-KO cells demonstrated significant changes in expression of genes related to rRNA processing, cell proliferation, and metabolic function, indicating metabolic reprogramming. Next, key genes were found to be downregulated in GSK3β-KO NK cells were upregulated in GSK3β-overexpressing NK cells from AML patients, confirming their clinical relevance. Lastly, cellular energetics were measured and it was observed that GSK3β-KO NK cells exhibited 150% higher spare respiratory capacity, a marker of metabolic fitness. These findings show a role for GSK3β in regulating NK cell metabolism.

An approach for studying human NK cell biology was described as combining NK cell expansion with CRISPR gene editing using Cas9 complexed with guide ribonucleoproteins (Cas9/RNP). Herein, this approach was applied to studying the transcriptional and functional role of GSK3β by generating CRISPR mediated GSK3β-KO NK cells, to avoid the confounding off-target effects of small-molecule drug inhibition. To confirm the clinical relevance of the differentially expressed genes found in GSK3β-KO NK cells, they were compared with NK cells from patients with AML-NK, which was previously shown to have elevated GSK3β. Lastly, the metabolic alterations in NK cells induced by loss of GSK3β was investigated.

### Material and Methods

Patient samples: NK cells were isolated from peripheral blood of healthy donors (American Red Cross, Columbus, OH) or untreated AML (Ohio State University Leukemia Tissue Bank). All studies were approved through the Ohio State University Institutional Review Board, protocol number 2009C0019.

Tumor cell lines: HL60 (AML), Kasumi1 (AML), K562 (CML), DAOY (medulloblastoma), and MG63 (osteosarcoma) cell lines were purchased from the American Type Culture Collection (ATCC, Manassas, Virginia, U.S.). U373 (glioblastoma) was kindly donated by Kevin Cassady (Nationwide Children's Hospital, Columbus, Ohio, USA). CSTX002 feeder cells (K562 genetically modified to express 4-1BBL and membrane-bound IL-21, referred to hereafter as FC-21) was generated.

NK cell isolation and expansion: Buffy coats from healthy volunteer red blood cell donations were obtained from the American Red Cross (Columbus, OH, USA), from which NK cells were isolated using RosetteSep™ Human NK Cell Enrichment Cocktail (Stem Cell Technologies, 15065, Vancouver, BC, Canada). Purified NK cells (CD3^{negative}/CD56^{positive}) were stimulated weekly for two weeks with irradiated CSTX002 feeder cells in AIM-V expansion medium supplemented with ICSR (CTS™AIMV™SFM/CTS™ Immune Cell SR, Thermo Fisher Scientific) and 50 IU of human recombinant IL-2 (rIL-2) (Novartis).

Generation of CRISPR-edited NK cells: GSK3β-KO NK cells were generated by electroporation of Cas9/RNP into NK cells at day 7 of expansion, targeting exon 5 of the GSK3β gene (5-CAGTATCAGGATCCAACAAG (SEQ ID NO: 1)).

NK function assays: Calcein-AM was used to evaluate cytotoxicity. Briefly, tumor cell targets were loaded with 2 ug/mL of Calcein-AM for 30 minutes. Cells were washed and incubated with WT or GSK3β-KO NK cells at multiple effector/target (E:T) ratios for 4 hours, as indicated in the figure legends.

Metabolic assays: Metabolic assays were performed. Briefly, we used Seahorse XF Cell Mito Stress Test Kit to measure the oxygen consumption rate (OCR) (Cat #103015-100, Agilent Technologies, Santa Clara, CA, United States) and Seahorse XF Glycolysis Stress Test Kit to measure extracellular acidification rate (ECAR) (Cat #103020-100, Agilent Technologies, Santa Clara, CA, United States). Cell-Tak-coated plates—Corning® Cell-Tak™—(Cat #354240, Bedford, Massachusetts) were used together with Agilent Extracellular Flux Pak (Cat #102416-100, Agilent Technologies, Santa Clara, CA, United States) on the Seahorse XFe96 analyzer (Agilent Technologies, Santa Clara, CA, United States). Expanded WT and GSK3βKO NK cells were preconditioned in XF RPMI at 37° C. in a non-CO2 incubator for 1 to 2 hours before the measurements. The medium was supplemented with 10 mM glucose and 1 mM L-glutamine with no phenol red at a pH of 7.35 to 7.4. OCR and ECAR were determined under the basal condition and after the addition of 1 mM oligomycin, 1.5 mM carbonyl cyanide-4-(trifluoromethoxy)phenylhydrazone (FCCP), 0.5 mM rotenone, and 0.5 mM antimycin A to the cell culture.

Antibodies: The following antibodies were used for flow cytometry: anti-GSK3β pS9-PE (Cat #130-106-964—Miltenyi Biotec), CD57 (Cat #130-111-964—Miltenyi Biotec). The following antibodies were used for Western Blot: GSK3β Rabbit mAb-27C10 (Cat #9315S—Cell Signaling—1:1000), ß-Actin Mouse mAb—8H10D10 (Cat #3700S—Cell Signaling—1:1000), Anti-Rabbit IgG HRP-linked (Cat #7074S—Cell Signaling—1:5000), Anti-Mouse IgG HRP-linked (Cat #7076S—Cell Signaling—1:5000).

Cytokine secretion: To induce cytokine secretion, NK cells at 2×10⁶/mL were stimulated with 10 μg/mL PHA. After 4 hours of incubation, supernatants were collected and kept in −80° C. On the day of the assay, the supernatants were thawed and measured in duplicate with Bio-Rad Bio-plex Pro Human Immunotherapy Panel 20-Plex (Cat #12007975) according to manufacturer's instructions. Data were acquired on Bio-rad Bio-Plex 200 system and analyzed with Bio-plex Manager software using curve fitting with logistic regression (5PL regression).

RNA-sequencing on non-expanded healthy and AML-NK: RNA-sequencing (RNA-seq) analysis was performed. Briefly, freshly sorted NK cells from normal donor peripheral blood (American Red Cross; n=3 donors) or newly diagnosed AML patients (Ohio State University Leukemia Tissue Bank; n=5; OSU IRB #2009C0019), were pelleted and total RNA was isolated using the Qiagen RNeasy Mini Kit (Qiagen). Directional poly-A RNA sequencing libraries were prepared and sequenced as 42-bp paired-end reads on an Illumina NextSeq 500 instrument (Illumina) to a depth of 33.2-48.0×10⁶ read pairs (Active Motif). Alignment to human genome (hg19 build) was done using TopHat. Transcriptome assembly and analysis was performed using Cufflinks and expression was reported as FPKM.

RNA-sequencing on expanded WT and GSK3β-KO NK cells: For RNA-seq on expanded WT and GSK3β-KO NK cells, RNA libraries were prepared using the TruSeq RNA Sample Preparation Kit (Illumina Inc.) and XX×10⁶ sequence reads (150 bp each) were generated per library using the Illumina HiSeq4000 platform (Institute for Genomic Medicine, Nationwide Children's Hospital). Reads were aligned and count tables were generated using Kallisto (v 0.43.1). Differential expression was then done using the Bioconductor package DeSeq2 (v 1.36.0). Volcano plots were generated using the package Glimma (v. 2.6.0) within R. Gene ontology (GO) was performed using GOrillia and subsequently visualized using REVIGO via their online web portal.

### Results

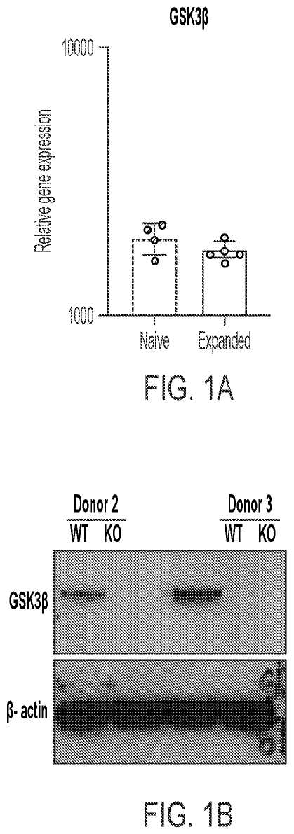

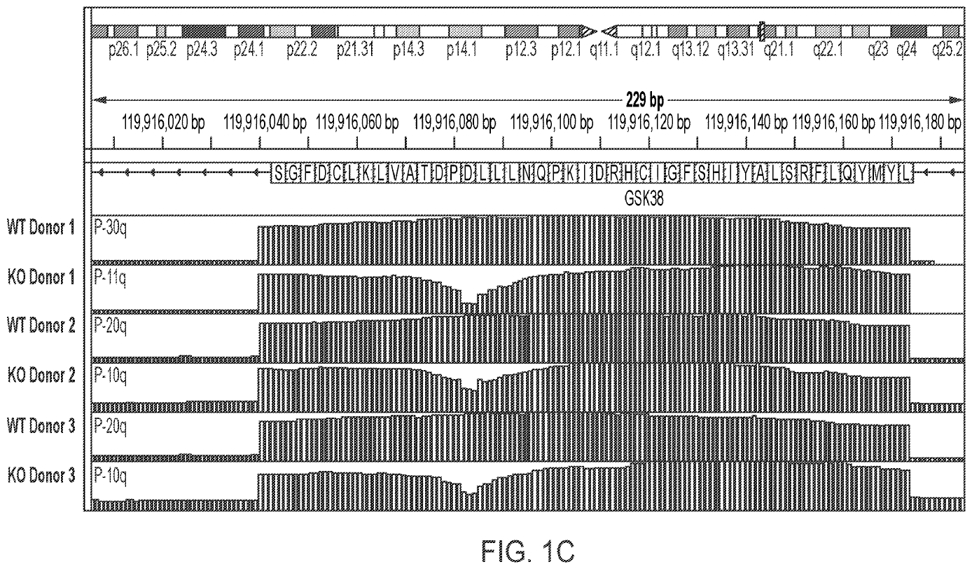

Deletion of GSK3β in FC-21 expanded NK cells. A gene editing approach for primary NK cells was previously described in which propagation with FC-21 feeder cells enhances DNA repair machinery and expands edited cells to large numbers. It was previously shown that GSK3β becomes overexpressed when NK cells are expanded in presence of soluble IL-15, and is associated with inhibition of their maturation and cytotoxic activity. Therefore, the relative gene expression of GSK3β in both naïve and FC-21-expanded NK cells was first evaluated to assess the stability of GSK3β in this model system. The expression level of GSK3β was similar between WT and expanded NK cells (FIG. 1A). To study the role of GSK3β and to avoid off-target effects of chemical inhibitors GSK3β-KO NK cells were generated and the knock-out efficiency at the protein level by western blot and at the mRNA level by RNA-sequencing was evaluated. The data demonstrated successful GSK3β gene deletion in FC-21 expanded NK cells (FIGS. 1B, 1C and FIG. 6).

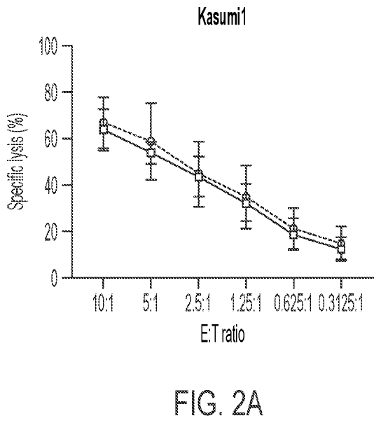

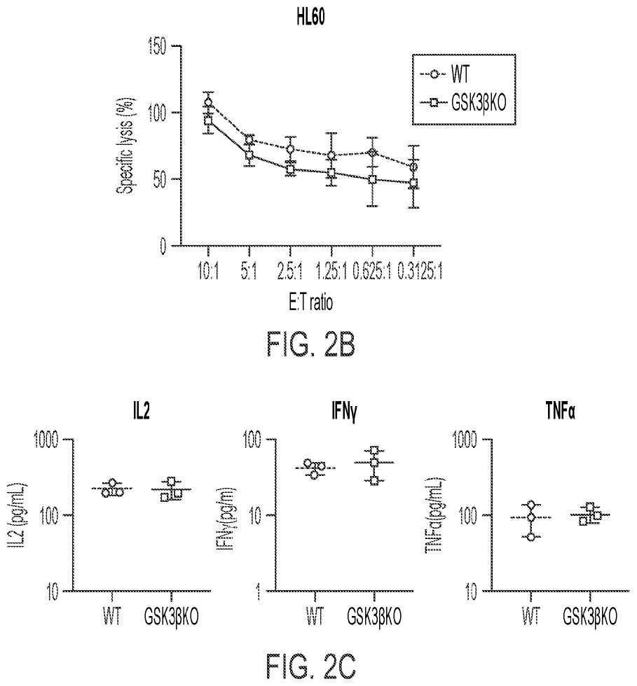

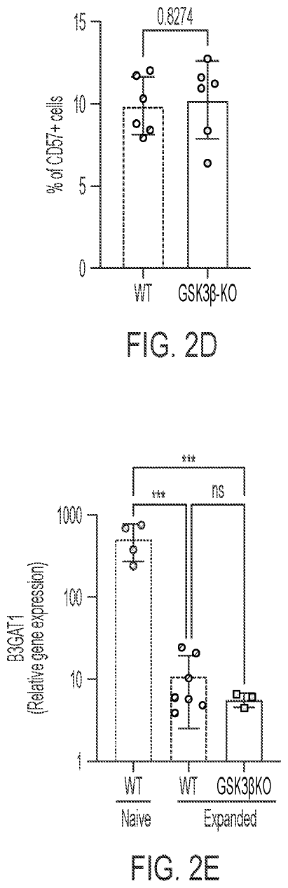

Deletion of GSK3β does not alter killing potency, cytokine secretion, or maturation of FC-21 expanded NK cells. Drug inhibition of GSK3β can improve NK cell killing against AML in NK cells with elevated GSK3β, such as those expanded with IL15 or from patients with AML. Therefore, the effect of knocking out GSK3β in FC-21 expanded NK cells was assessed based on their anti-AML activity. Unexpectedly, the deletion of GSK3β did not alter NK cell killing against AML cell lines HL60 and Kasumi1 in a standard 4-hour killing assay (FIGS. 2A and 2B). Additionally, GSK3β-KO NK cells showed similar killing potency as WT NK cell against K562 (CML), DAOY (medulloblastoma), and MG63 (osteosarcoma). In the previously reports, the improved killing in NK cells treated with GSK3β chemical inhibitors was also associated with increased cytokine secretion. Thus, cytokine secretion was evaluated in WT and GSK3β-KO and found that both WT and GSK3β-KO NK cells secrete high levels of IL-2, IFNγ and TNFα, and no difference was found between the two (FIG. 2C).

Additionally, it was previously reported that GSK3β drug inhibition drove maturation of IL-15 expanded NK cell, evidenced by CD57 expression (Cichocki, F., et al., GSK3 Inhibition Drives Maturation of NK Cells and Enhances Their Antitumor Activity. Cancer Res, 2017. 77(20): p. 5664-5675). Thus, CD57 expression was analyzed on both FC-21 expanded WT and GSK3β-KO NK cells by flow cytometry and found no difference between them (FIG. 2D). FC-21 expanded NK cells were then examined for their expression of B3GAT1, the gene encoding the enzyme responsible for catalyzing the CD57 carbohydrate epitope. Interestingly, B3GAT1 was 30-fold downregulated in FC-21 expanded NK cells compared to naïve NK cells, and this low expression was not altered by GSK3β deletion (FIG. 2E). This explains the low CD57 surface expression in both WT and gene edited cells, and also shows that B3GAT1 is not directly regulated by GSK3β and that other elements may be involved in regulating the expression of CD57 in NK cells.

### Transcriptional Changes in GSK3β-KO NK Cells

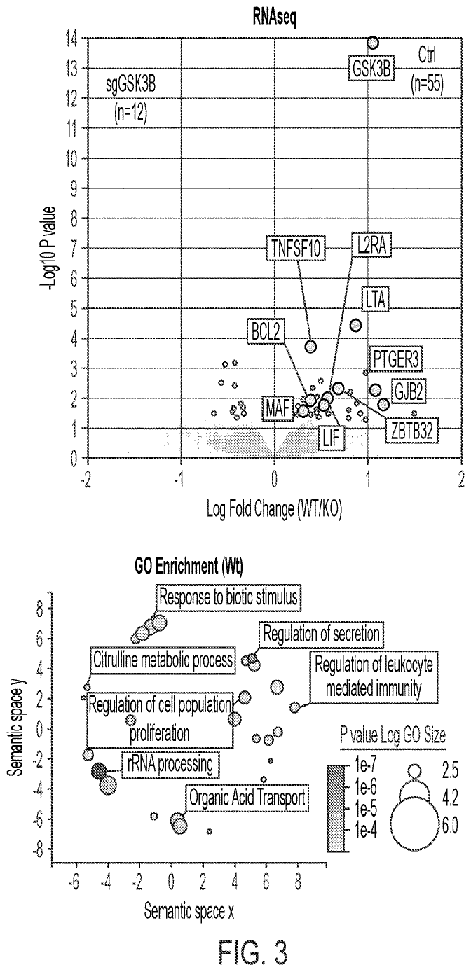

To study the role of GSK3β in NK cells, bulk RNA-seq was performed on WT or GSK3β-KO NK cells after expansion with FC-21. Differential gene expression analysis via Deseq2 revealed 55 genes significantly upregulated (adjusted P value <0.05, Paired Deseq2 test) in WT NK cells and 12 genes significantly upregulated in the GSK3β-KO cells. Thus, the majority of changes detected were of loss of expression concurrent with the loss of GSK3β expression (FIGS. 3A and 3C), which was consistent with the read drop across exon 6 at the GSK3β locus seen in FIG. 1C.

Gene ontology (GO) analysis of the 55 WT-specific transcripts was then used to determine what expression programs were regulated by GSK3β. GO results were then visualized via REVIGO, which clusters redundant categories for biological interpretation (FIG. 3B). Unique GO clusters included those for ribosomal RNA (rRNA) processing, secretion, metabolic processes, and proliferation (FIG. 3B). For example, GSK3β-KO lost transcription of genes involved within NK cell homeostatic renewal, such as the high affinity IL2 trimeric receptor, encoded by IL2RA, and the survival gene BCL2 (FIG. 3C). Downregulation of genes that encode for TNFα effectors, namely leukemia inhibitory factor (LIF) and TNFSF10 (encoding TRAIL), were observed within GSK3β-KO. Additional expression analysis showed deregulation of global biosynthesis as some genes involved in ribosomal RNA processing were lost (e.g. WDR3, WDR74, see FIG. 3C) and those involved in mitochondrial function were gained (e.g. MT-ND4 and MT-ND2, see FIG. 3C). Together, transcriptome analysis showed a role of GSK3β in regulating NK cell homeostasis, effector function, and metabolism.

### Deletion of GSK3β Leads to Metabolic Reprogramming of NK Cells.

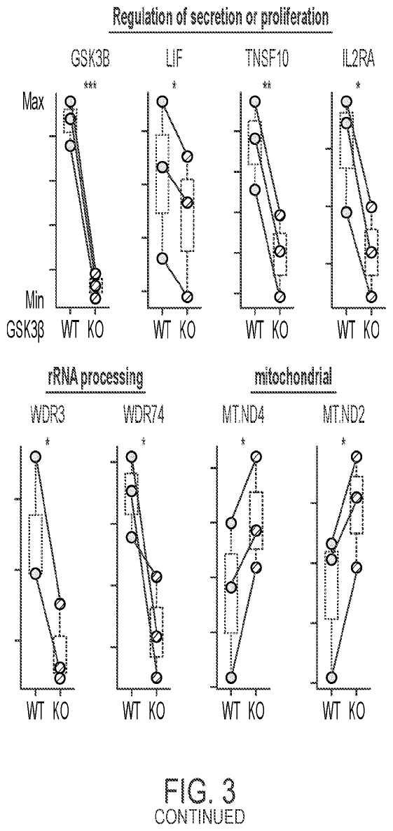

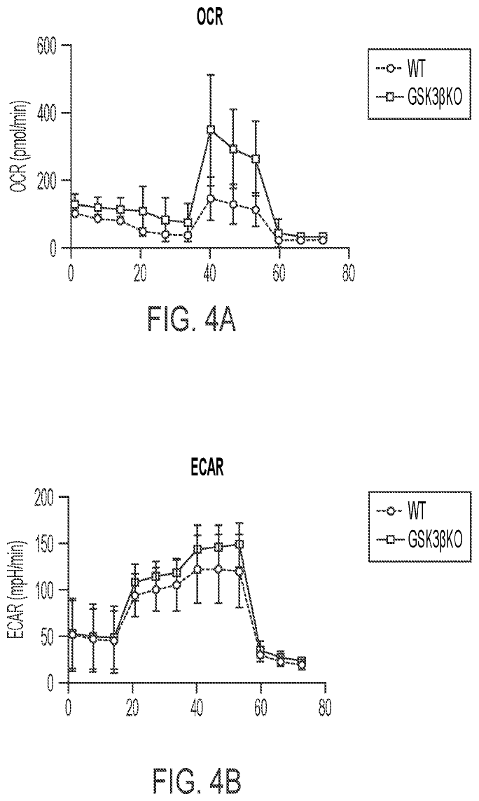

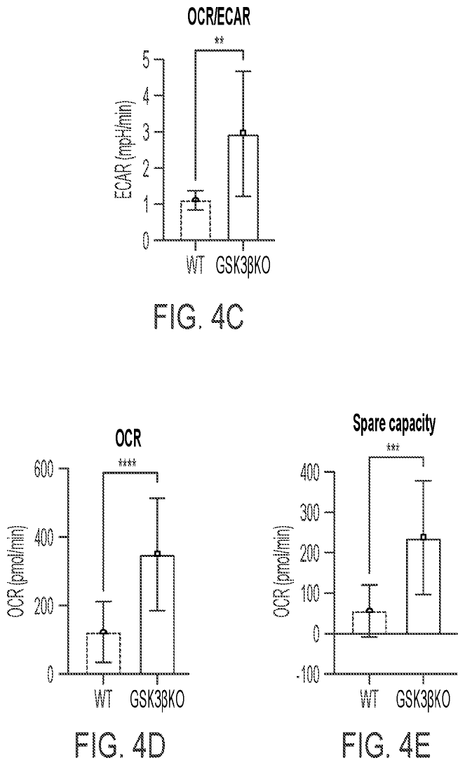

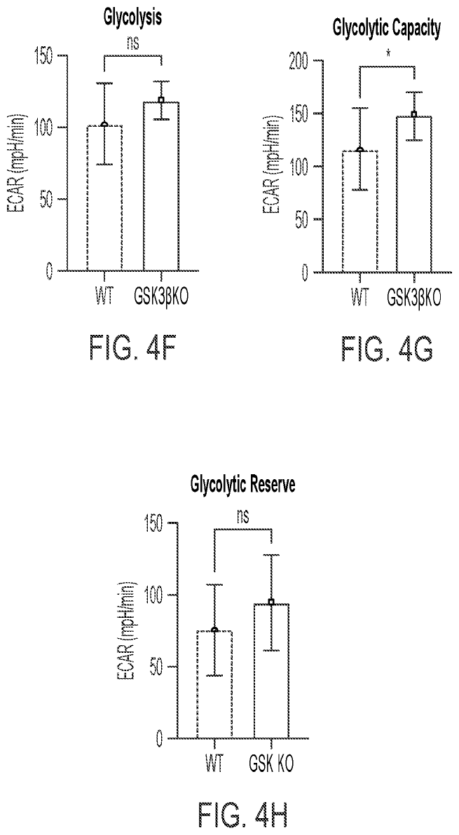

To explore the effect of upregulation of the mitochondrial genes identified by RNA-seq, the cellular metabolism of WT and GSK3β-KO NK cells was examined by assessing both mitochondrial (FIGS. 4A, 4D and 4E) and glycolytic stress (FIGS. 4B, 4F, 4G, and 4H). GSK3β-KO NK cells presented an overall shift towards oxidative metabolism with higher OCR and spare respiratory capacity (FIGS. 4A, 4B, 4C, 4D, and 4E). Spare respiratory capacity was previously described as pivotal for memory T cell formation and as a reliable indicator of metabolic fitness. Additionally, GSK3βKO NK cells also showed modestly higher ECAR, glycolytic capacity, and glycolytic reserve than WT NK cells, indicating that the increase in oxidative phosphorylation was not compensatory for decreased glycolysis. Together this shows that deletion of GSK3β induces NK cells to enhance overall metabolic capacity, which is beneficial for sustaining antitumor activity.

GSK3β is Highly Expressed in NK Cells at Stage 5 and 6 of Maturation from AML Patients

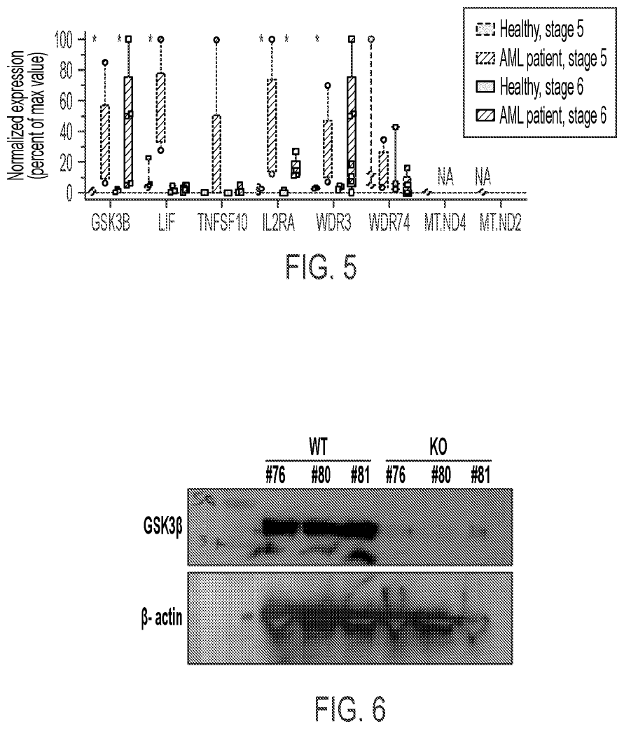

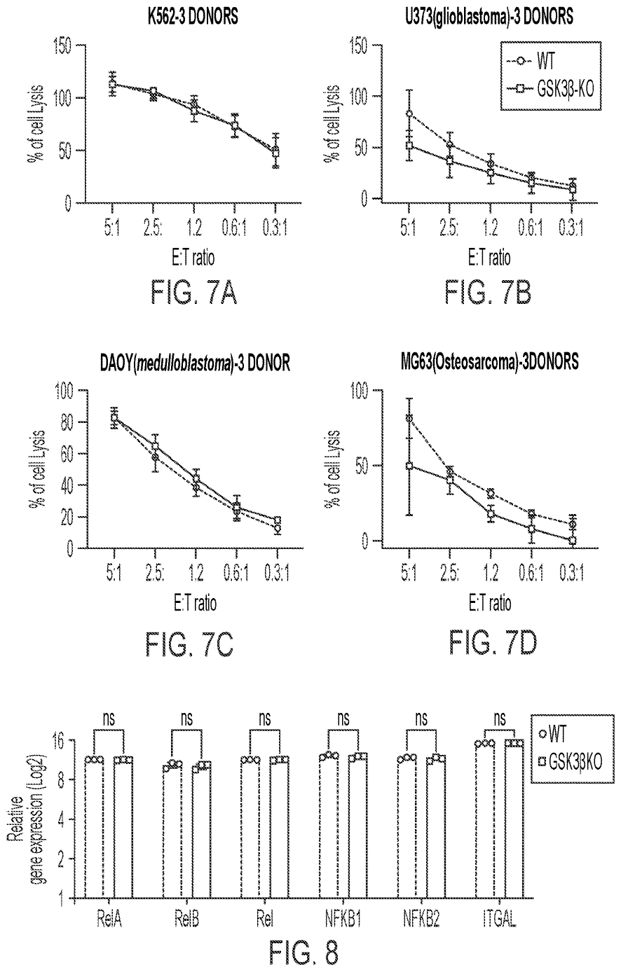

To further study the role of GSK3β in NK cells, transcriptional changes in NK cells from AML patients was examined which have been shown to overexpress GSK3β, in order to validate key genes identified as differentially expressed in GSK3β-KO NK cells. To correct for confounding by differences in NK cell maturation, the expression of level of these genes in NK maturation stages 5 and 6 were studied separately. First, GSK3β was confirmed to be statistically elevated in NK cells from AML patients when compared to healthy donor NK cells, for both stage 5 and stage 6 cells. The relative expression of the key genes identified in GSK3β-KO NK cells (FIG. 5), and found strong concordance of downregulated genes was then evaluated in GSK3β-KO NK cells being upregulated in GSK3β-overexpressing NK cells from patients with AML. LIF was only overexpressed in stage 5 but not in stage 6. IL2RA expression was significantly increased in both AML-NK5 and AML-NK6 when compared to healthy NK cells. No difference was observed in expression level of WDR74, but WDR3 was significantly increased in AML-NK5. The mRNA transcripts for MT-ND4 and MT-ND2 were not identified in any of these samples, despite finding MT-ND4 and MT-ND2 transcripts at high levels in the GSK3-KO experiments.

### DISCUSSION

NK cells play an important role in immunosurveillance and preventing tumor development and progression. NK cells from patients with AML exhibit great defects in both numbers and functional activities, unable to control AML development, progression and relapse. Even though phenotypic changes have been well described, specific molecular explanations for these dysfunctions are still needed. It was previously reported that the high expression of GSK3β on AML NK cells profoundly impacted NK cells killing ability (Parameswaran, R., et al., Repression of GSK3 restores NK cell cytotoxicity in AML patients. Nat Commun, 2016. 7: p. 11154). Herein, it was demonstrated that the enhanced expression of GSK3β is present in NK cells at both stage 5 and 6 of development in patients with AML. There was previously limited evidence on the biologic mechanisms of GSK3β-mediated dysfunction in NK cells. Cas9/RNP gene editing was used to generate GSK3β-KO NK cells to enable precise gene-level understanding of GSK3β effects on primary human NK cells, which revealed novel information on the impact of GSK3β on transcriptional regulation of homeostasis, effector function, and metabolism. The expansion of NK cells on FC-21 was shown to not increase GSK3β expression levels as was observed in IL15-expanded NK cells, which reduces the need for GSK3β inhibition in adoptive NK cell therapy.

Deletion of GSK3β resulted in increased mitochondrial respiratory capacity that was associated with an increase in Complex 1 genes, MT-ND2 and MT-ND4, which favor OXPHOS metabolism. Defined as “The Most Complex Complex”, Complex 1, also known as NADH dehydrogenase, is composed of 46 subunits, seven of which (including MT-ND2 and MT-ND4) are encoded in the mitochondrial genome. Complex 1 catalyzes electrons transfer from NADH through the respiratory chain, using ubiquinone as an electron acceptor. Furthermore, the expression of mitochondrial respiratory complex 1 was previously associated with gain in mitochondrial oxidative phosphorylation (OXPHOS) activity. Additionally, targeting OXPHOS with a complex I inhibitor decreased OXPHOS in pancreatic cancer cells reinforcing the correlation between OXPHOS and Complex 1. In line with previously published evidence, this data showed that GSK3β deletion resulted in increased OXPHOS, with higher maximum and spare respiratory capacities. The edited cells also shift their metabolic profile more towards OCR than ECAR which can improve ATP generation and provide more energy to the cells essential for effector function. It was previously demonstrated that NK cells from both leukemic mice and patients with AML displayed similar metabolic defects (Bou-Tayeh, B., et al., Chronic IL-15 Stimulation and Impaired mTOR Signaling and Metabolism in Natural Killer Cells During Acute Myeloid Leukemia. Front Immunol, 2021. 12: p. 730970). Several groups showed that NK cells from AML patients or expanded with IL15 have high levels of GSK3β and that chemical inhibition of GSK3β can improve their antitumor activity through an increase in NFKB signaling molecules (RELA, RELB, c-REL, NFKIBA). However, the data herein showed that FC21 expansion of NK cells does not increase GSK3β levels, and therefore its deletion did not alter their cytotoxicity, at least in the short-term 4-hour killing assays used.

NK cells with high GSK3β have been shown to express low levels of CD57. A benefit of using GSK inhibitors in these NK cells was to increase CD57 expression level showing maturation to an adaptive memory-like phenotype. This protein is recognized as a maturation/senescence marker and its frequency increases with age and it has been reported to be missing or dim on fetal and infant NK cells. The CD57 expression, denoting terminal differentiation of NK cells, should be beneficial from an immunotherapy standpoint, as several clinical studies reported that high expression of CD57 on NK cells is associated with better clinical outcome. It has been shown that drug inhibition of GSK3β resulted in increased CD57 expression. Of note, CD57 is not a DNA-encoded protein, but rather a carbohydrate epitope catalyzed by B3GAT1. The present example showed B3GAT1 levels decreased in FC-21 expanded NK cells, but both CD57 and B3GAT1 remain unchanged after the deletion of GSK3β. This shows that GSK3β does not directly regulate B3GAT1, or therefor CD57, indicating other regulators and/or pathways control the CD57 expression associated with maturation and memory. Taken together, GSK3β is shown to be a negative metabolic regulator in NK cells.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A method of generating a glycogen synthase kinase 3 beta (GSK3β) deficient natural killer (NK) cell, the method comprising:
a. isolating the NK cell;
b. deleting GSK3β in the NK cell using a gene editing system; and
c. expanding and stimulating the NK cell with feeder cells comprising membrane bound (mb) IL21 on the surface of the feeder cells,
wherein the GSK3β deficient NK cell comprises decreased GSK3β expression and increased cellular metabolism relative to a control.

2. The method of claim 1, wherein the gene editing system comprises a CRISPR/Cas9 gene editing system.

3. The method of claim 1, wherein the gene editing system comprises introducing a Cas9/ribonucleoprotein (RNP) complex into the NK cell.

4. The method of claim 1, wherein the GSK3β deficient NK cell is stimulated with feeder cells at least once per week.

5. The method of claim 1, wherein the feeder cells further comprise membrane bound 4-1BBL.

6. The method of claim 1, wherein the GSK3β deficient NK cell is stimulated with feeder cells for at least two weeks.

7. The method of claim 1, wherein the feeder cells are K562 feeder cells.

8. (canceled)

9. The method of claim 1, wherein the GSK3β deficient NK cells are supplemented with at least 50IU of an IL-2.

10. The method of claim 9, wherein the IL-2 is a human recombinant IL-2 (rIL-2).

11. (canceled)

12. A natural killer (NK) cell modified by the method of claim 1.

13. A method of treating cancer in a subject, the method comprising administering to the subject a composition comprising a glycogen synthase kinase 3 beta (GSK3β) deficient natural killer (NK) cell and a pharmaceutically acceptable carrier, wherein the GSK3β deficient NK cell comprises increased cellular metabolism and increased antitumor activity.

14. The method of claim 13, wherein GSK3β has been deleted from a NK cell using a CRISPR/Cas9 gene editing system.

15. (canceled)

16. The method of claim 13, wherein the CRISPR/Cas9 gene editing system comprises introducing a Cas9/ribonucleoprotein (RNP) complex into the NK cell.

17. The method of claim 13, wherein the GSK3β deficient NK cell is expanded with feeder cells comprising membrane (mb) IL21 on the surface of the feeder cells.

18. The method of claim 13, wherein the GSK3β deficient NK cell is stimulated with feeder cells at least once per week.

19. The method of claim 13, wherein the feeder cells further comprise membrane bound 4-1BBL.

20. The method of claim 13, wherein the GSK3β deficient NK cell is stimulated with feeder cells for at least two weeks.

21. The method of claim 13, wherein the feeder cells are K562 feeder cells.

22. (canceled)

23. The method of claim 13, wherein the GSK3β deficient NK cells are supplemented with at least 50IU of an IL-2.

24. The method of claim 23, wherein the IL-2 is a human recombinant IL-2 (rIL-2).

25-26. (canceled)
